(19) United States
(12) Patent Application Publication
(10) Pub. No.: US 2015/0293012 A1
(43) Pub. Date: Oct. 15, 2015

(54) RECEPTACLE AND SYSTEM FOR OPTICALLY ANALYZING A SAMPLE WITHOUT OPTICAL LENSES

(71) Applicant: FRAUNHOFER-GESELLSCHAFT ZUR FÖRDERUNG DER ANGEWANDTEN FORSCHUNG E.V., München (DE)

(72) Inventors: Daniel Hans Rapoport, Luebeck (DE); Tim Becker, Bad Schwartau (DE); Charli Kruse, Herrnburg (DE)

(21) Appl. No.: 14/441,834

(22) PCT Filed: Nov. 9, 2012
(86) PCT No.: PCT/EP2012/004668

§ 371 Date: May 8, 2015

(51) Int. Cl. G01N 21/03; G01N 33/483; G01N 15/14; G02B 21/16; G02B 21/36; G01N 21/64

(52) U.S. Cl. CPC G01N 21/0303; G02B 21/361; G01N 21/6486; G01N 21/6458; G01N 15/1436; G02B 21/16; G01N 33/4833; G01N 2021/6471; G01N 2201/068; G01N 2201/062; G01N 2201/022

(57) ABSTRACT

The invention relates to a receptacle (4) for receiving a sample (5) during an optical analysis of the sample (5), wherein the receptacle (4) comprises a bottom (19) which is at least partially transparent so that the sample (5) within the receptacle (4) can be optically analyzed by an image sensor (6) from below the bottom (19) and wherein the bottom (19) is very thin thereby improving contrast and sharpness of the images generated by the image sensor (6). Further, the invention relates to a system for optically analyzing a sample (5).

### FIELD OF THE INVENTION

The invention relates to a receptacle for receiving a sample, e.g. a biological sample like a cell culture, during an optical analysis of the sample. Further, the invention relates to a system for optically analyzing a sample, e.g. a biological sample like a cell culture.

### BACKGROUND OF THE INVENTION

The state of the art comprises the so-called time-lapse microscopy which is used for life-cell imaging. The conventional time-lapse microscopy systems comprise an optical microscope, a digital camera, a computer software and an incubator to control the cellular environment of the sample. However, the conventional time-lapse microscopy systems are very expensive and complex. Further, it is difficult to integrate time-lapse microscopy systems into working environments in laboratories since they are too big and too complex for an integration into a conventional incubator. Finally, it is difficult to use the conventional time-lapse microscopy systems in connection with automated cell cultures (“cell farms”), which are needed in clinical applications.

Further, the state of the art comprises so-called Petri dishes which are receptacles for receiving a sample during an optical analysis of the sample, for example during the aforementioned time-lapse microscopy.

Moreover, a so-called ePetri dish is disclosed in Zheng et al.: “The ePetri dish, an on-chip cell imaging platform based on sub-pixel perspective sweeping microscopy (SPSM)”, Proceedings of the national Academy of Sciences of the United States of America (PNAS) 2011. According to this idea, cell cultures are directly placed on the surface of a CMOS image sensor without any optical lenses in between. However, the CMOS image sensor is contaminated by the direct contact with the cell culture. Therefore, each measurement of a cell culture needs a new CMOS image sensor or a thorough cleaning of a used CMOS image sensor.

### SUMMARY OF THE INVENTION

Therefore, it is an object of the invention to provide an improved system for optically analyzing a sample, e.g. a biological sample like a cell culture.

Further, it is an object of the invention to provide an improved receptacle which is suitable for the novel system for optically analyzing a sample.

These objects are achieved by a system and a receptacle according to the independent claims.

Firstly, the invention provides a novel receptacle for receiving a sample during an optical analysis of the sample, wherein the receptacle comprises a bottom which is at least partially transparent, so that the sample within the receptacle can be optically analyzed by an image sensor from below the bottom.

In contrast to the conventional Petri dishes, the receptacle according to the invention comprises a very thin bottom with a thickness of less than 500 μm, 200 μm, 150 μm or even less than 120 μm. The thin bottom of the receptacle advantageously allows the use of the so-called “shadow imaging” for optically analyzing the sample within the receptacle. During the so-called shadow imaging, the receptacle with the sample is placed directly on the photosensitive area of an image sensor (e.g. a CCD sensor or a CMOS sensor) without any optical lens between the receptacle and the image sensor. It is important to have a very low thickness of the bottom of the receptacle in order to improve contrast and sharpness of the shadow imaging. The principles of the so-called shadow imaging are explained in Zheng et al.: “The ePetri dish, an on-chip cell imaging platform based on sub-pixel perspective sweeping microscopy (SPSM)”, Proceedings of the national Academy of Sciences of the United States of America (PNAS) 2011. Therefore, the content of this publication is incorporated by reference herein.

Further, the thin bottom of the receptacle also allows gas diffusion through the bottom of the receptacle, so that it is not necessary to provide a conventional septum for CO2-exchange (carbonate buffer).

Moreover, the receptacle according to the invention can be functionalized in order to improve the optical contrast of the imaging. In one embodiment, an upper polarization filter is arranged above the sample between the sample and a light source illuminating the sample within the receptacle from above. Further, a lower polarization filter is arranged below the sample between the sample and the image sensor viewing the sample from below. Moreover, an optical waveguide structure is arranged between the upper polarization filter and the lower polarization filter. The upper polarization filter and the lower polarization filter are aligned perpendicular to each other thereby restricting the light received by the image sensor from the light source to specific optical modes thereby achieving an improvement of the optical contrast in comparison to conventional imaging methods.

The upper polarization filter can be arranged in a cover of the receptacle, while the lower polarization filter can be arranged in the bottom of the receptacle. Further, the aforementioned waveguide structure can also be arranged in the bottom of the receptacle on the surface facing the sample.

In another embodiment of the invention, an upper color filter is arranged above the sample between the sample and a light source illuminating the sample from above, wherein the wavelength of the illumination from the light source is preferably within the passband of the upper color filter, so that the illumination from the light source passes through the upper color filter. Further, a lower color filter can be arranged below the sample between the sample and the image sensor viewing the sample from below, wherein the wavelength of the light emitted by the sample in response to the illumination by the light source is preferably within the passband of the lower color filter, so that the light emitted by the sample passes the lower color filter.

The upper color filter can be arranged in the cover of the receptacle, while the lower color filter can be arranged in the bottom of the receptacle.

In the afore-mentioned embodiment of the receptacle comprising upper and lower color filters, the upper side of the bottom of the receptacle is preferably coated with a pH-sensitive fluorescent dye emitting light in response to the illumination by the light source. Those parts of the pH-sensitive fluorescent dye not in contact with the sample emit light at an emission wavelength outside the passband of the lower color filter, while those parts of the pH-sensitive fluorescent dye in contact with the sample are pH-shifted by the sample thereby shifting the emission wavelength of the pH-sensitive fluorescent dye, wherein the shifted emission wavelength of the pH-sensitive fluorescent dye is within the passband of the lower color filter. In other words, the light source illuminates the sample through the upper color filter with the excitation wavelength of the pH-sensitive fluorescent dye and the image sensor detects fluorescence in those parts which are covered by the sample. In this connection, it should be noted that the passband of the upper color filter is matched to the excitation wavelength of the pH-sensitive fluorescent dye, while the passband of the lower color filter is matched to the shifted emission wavelength of the pH-sensitive fluorescent dye.

In another embodiment of the invention, the receptacle comprises at least one calibration element for optical calibration of the receptacle. The calibration element can be used for determining the transfer function of the optical system allowing a more accurate analysis of the sample within the receptacle.

In a preferred embodiment of the invention, a light source is integrated in the receptacle for illuminating the sample within the receptacle from above. For example, the light source can be arranged at least partially in the cover of the receptacle. Further, it should be noted that the light source is preferably point-shaped thereby improving the optical contrast and sharpness of the images of the sample.

In one embodiment of the invention, the light source comprises a lamp, for example a light emitting diode (LED) or an organic light emitting diode (OLED), which is preferably arranged in the cover of the receptacle. In another embodiment of the invention, the light source comprises a hole in the cover of the receptacle, wherein the sample is illuminated from above through the hole in the cover of the receptacle.

Alternatively, the light source comprises a reflecting element above the sample, particularly at the lower side of the cover, and a lamp for illuminating the reflecting element from below, wherein the reflecting element is preferably shaped as a circle or as a half-sphere.

Further, it should be noted that the invention also claims protection for a system for optically analyzing a sample, wherein the system according to the invention comprises an image sensor with a photosensitive area with a plurality of photosensitive pixels, particularly a CCD sensor or a CMOS sensor.

Moreover, the system according to the invention comprises a receptacle for receiving the sample during analysis of the sample, wherein the receptacle is preferably designed as illustrated above. In contrast to the initially mentioned conventional time-lapse microscopy, the receptacle is arranged directly on the photosensitive area of the image sensor without an optical lens between the receptacle and the image sensor. Therefore, the system according to the invention preferably allows the so-called shadow imaging without complex optics.

It should be noted that air gaps between the lower surface of the bottom of the receptacle and the photosensitive area of the image sensor cause multiple reflections and deteriorate the quality of the imaging process. These air gaps can be avoided by pressing the receptacle onto the photosensitive area. Therefore, the system according to the invention preferably comprises a pressing mechanism for pressing the receptacle onto the photosensitive area of the image sensor thereby avoiding air gaps between the receptacle and the image sensor.

Further, air gaps between the bottom of the receptacle and the photosensitive area of the image sensor can also be avoided by at least partially filling these gaps with a liquid, preferably an immersion oil or a polymer film.

Moreover, the optical resolution can be improved by moving the image sensor relative to the receptacle perpendicular to the optical axis, i.e. in the plane of the photosensitive area of the image sensors. Then, several images can be taken in different positions of the receptacle relative to the image sensor. These single images can then be used for generating an image with an improved optical resolution. Therefore, the system according to the invention preferably comprises an actuator, e.g. a piezo actuator for moving the image sensor relative to the receptacle in the plane of the photosensitive area of the image sensor in order to increase the optical resolution of the measurement. In this connection, it should be noted that the amplitude of the relative movement between the receptacle and the image sensor is preferably smaller than the distance between adjacent pixels of the image sensor.

Further, the optical resolution can also be improved by varying the direction of illumination of the sample within the receptacle. For example, the direction of illumination can be varied by providing an array of lamps or mirrors, wherein the lamps or mirrors are located at different places above the sample for successively illuminating the sample from different angles.

The optical system according to the invention preferably allows an analysis of a quite large area of a sample. Therefore, the photosensitive area of the image sensor is preferably larger than 200 mm² or 1 cm².

Further, it should be noted that the light flux passing through the sample is much smaller than the light flux passing through the sample in a light microscope so that the light exposure of the sample is reduced. This can be important for a long-term analysis of living cells which can be damaged by an intensive long-term illumination.

The concept of the invention allows small dimensions of the entire system so that the entire system can be integrated into existing working environments in laboratories. For example, the system can be integrated in a conventional incubator.

Finally, the invention preferably comprises an evaluation unit for an image-based evaluation of the images recorded by the image sensor. The evaluation unit preferably performs at least one of the following steps:
Detection of biological cells in the images recorded by the image sensor.
Tracking of the position of the biological cells within the image.
Detecting apoptosis of the biological cells.
Detecting necrosis of the biological cells.
Detecting mitosis of the biological cells.
Determination of the image entropy of the image recorded by the image sensor.
Determination of cytometric data, e.g. total number of the biological cells within the image, cell proliferation of the cells within the image, frequency of mitosis among the biological cells within the image, morphological parameters of the biological cells, particularly size, length and/or brightness of the biological cells or duration of a cell cycle.

The details of the evaluation unit are also explained in Rapoport et al.: “A novel validation algorithm allows for automated cell tracking and the extraction of biologically meaningful parameters”, PLoS ONE 6(11): e27315.doi:10.1371/—journal.pone.0027315. Therefore, the content of this publication is incorporated by reference herein.

The invention and its particular features and advantages will become more apparent from the following detailed description considered with reference to the accompanying drawings.

### BRIEF DESCRIPTION OF THE DRAWINGS

FIG. 1 is a scheme illustrating the system according to the invention for analysis of a biological sample.

FIG. 2 is a cross section of a receptacle according to the invention comprising a point-shaped light source for illumination of the sample from above.

FIG. 3 is a modification of the receptacle according to FIG. 2 comprising a mirror in the cover for illuminating the sample.

FIG. 4 shows a cross section through another modification of a receptacle comprising polarization filters both in the cover and in the bottom of the receptacle.

FIG. 5 is a cross section through another modification of the embodiment according to FIG. 3 comprising a calibration element for optical calibration of the receptacle.

FIG. 6 shows a cross section through another modification of a receptacle comprising a hole in the cover for illuminating the sample through the hole.

FIG. 7A shows an air gap between the bottom of the receptacle and the image sensor.

FIG. 7B shows the gap filled with a liquid in order to avoid multiple reflections.

FIG. 8 shows a flowchart illustrating the operating procedure of the system according to the invention.

### DETAILED DESCRIPTION OF THE DRAWINGS

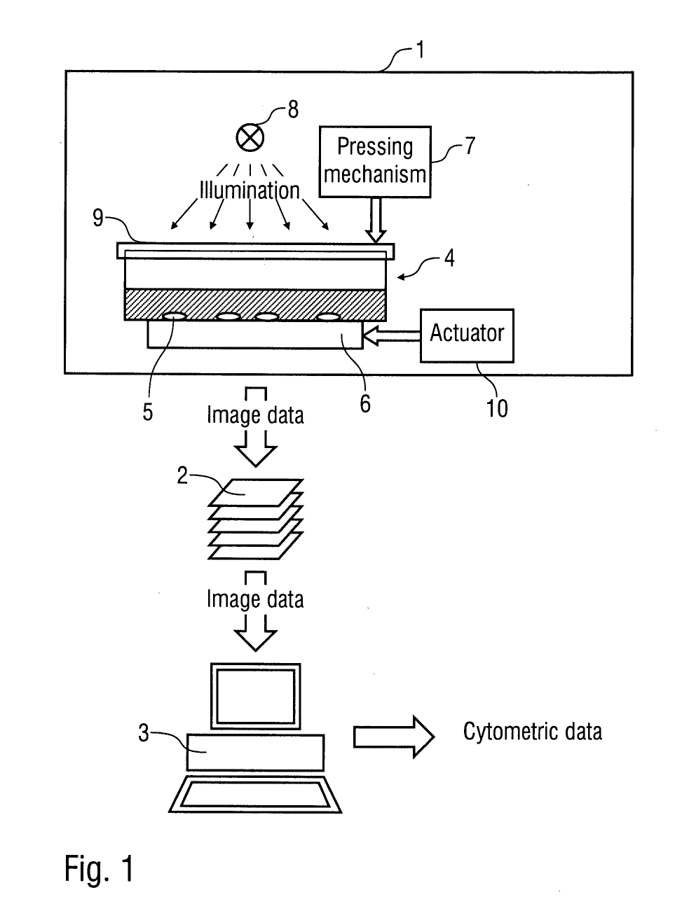

FIG. 1 illustrates a system according to the invention for optically analyzing a biological sample, for example a cell culture. The system comprises an image acquisition system 1 for generating optical images 2 of the sample and an evaluation unit 3 for analyzing the images 2 and generating cytometric data.

The image acquisition system 1 comprises a receptacle 4 which includes an optical sample 5 to be analyzed, wherein the receptacle 4 comprises a transparent bottom so that the sample 5 within the receptacle 4 can be optically analyzed by an image sensor 6 from below the receptacle 4 by “shadow imaging” as explained in Zheng et al.: “The ePetri dish, an on-chip cell imaging platform based on subpixel perspective sweeping microscopy (SPSM)”, Proceedings of the national Academy of Sciences of the United States of America (PNAS) 2011. Therefore, the content of this publication is incorporated by reference herein.

Further, it should be noted that the receptacle 4 comprises a transparent thin bottom with a thickness of less than 150 μm.

Firstly, this allows the co-called shadow imaging wherein the receptacle 4 is directly placed on the photosensitive area of the image sensor 6 without any lenses between the receptacle 4 and the image sensor 6. The thin bottom of the receptacle 4 results in an improved contrast and sharpness of the images 2 generated by the image sensor 6.

Further, the thin bottom of the receptacle 4 allows gas diffusion through the bottom, so that it is not necessary to provide a conventional septum for CO2-exchange.

Moreover, the image acquisition system 1 comprises a pressing mechanism 7 which presses the receptacle 4 onto the photosensitive area of the image sensor 6 thereby minimizing any air gaps between the lower surface of the bottom of the receptacle 4 and the photosensitive area of the image sensor 6. This is important since any air gaps between the receptacle 4 and the image sensor 6 cause multiple reflections thereby impairing the quality of the images 2.

Further, the image acquisition system 1 comprises a point-shaped light source 8 which is arranged above a removable cover 9 of the receptacle 4, so that the light source 8 illuminates the sample 5 within the receptacle 4 from above.

Moreover, the image acquisition system 1 comprises an actuator 10 (e.g. a piezo actuator) which moves the image sensor 6 relative to the receptacle 4 in the plane of the photosensitive area of the image sensor 6, i.e. perpendicular to the optical axis. Then, the image sensor 6 takes several images of the sample 5 in different positions of the image sensor 6 relative to the receptacle 4. This allows the evaluation unit 3 to calculate a resulting image with a higher optical resolution. In other words, the sub-pixel movements of the image sensor 6 relative to the receptacle 4 improve the effective optical resolution.

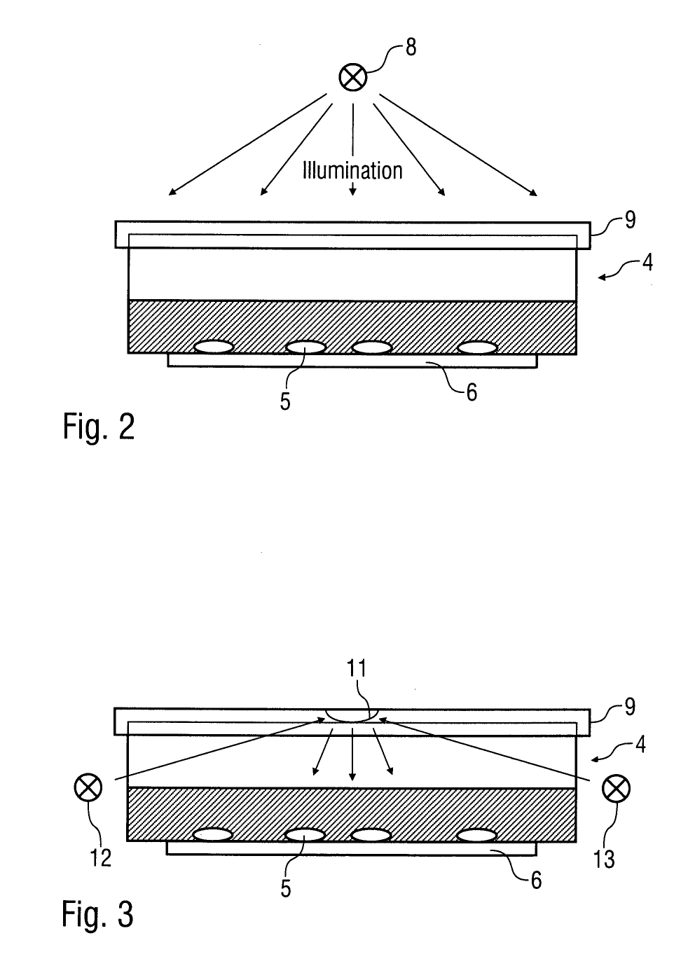

FIG. 2 shows a cross section through the receptacle 4 of the image acquisition system 1 of FIG. 1 along with the point-shaped light source 8.

FIG. 3 shows a modification of FIG. 2 so that reference is made to the above description, wherein the same reference signs are used to designate corresponding details.

Instead of the point-shaped light source 8, the embodiment of FIG. 3 comprises a reflecting element 11 (i.e. a mirror) which is arranged on the lower side of the cover 9 of the receptacle 4, wherein the reflecting element 11 is illuminated by two light sources 12, 13 which are arranged on opposite sides of the receptacle 4. Therefore, the sample 5 within the receptacle 4 can be illuminated from different directions either by the light source 12 or by the light source 13. The image acquisition system 1 takes images of the sample 5 with different directions of illuminations, which allows the evaluation unit 3 to calculate a resulting image with an improved optical resolution.

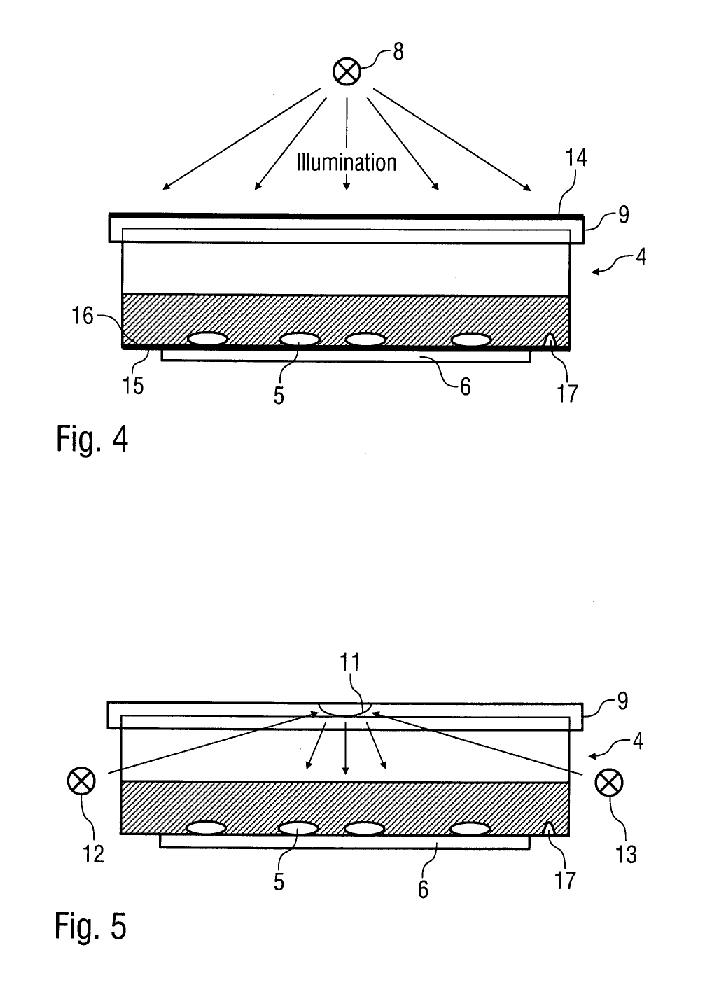

FIG. 4 shows another modification of the receptacle 4 as shown in FIG. 2, so that reference is made to the above description, wherein the same reference signs are used to designate corresponding details.

In this embodiment of the invention, an upper polarization filter 14 is arranged in the cover 9 of the receptacle 4. Further, a lower polarization filter 15 is arranged in the bottom of the receptacle 4, wherein the upper polarization filter 14 and the lower polarization filter 15 are aligned perpendicular to each other. Further, an optical waveguide structure 16 is applied to the upper surface of the bottom of the receptacle 4. The combination of the lower and upper polarization filters 14, 15 and the optical waveguide structure 16 improves the optical contrast as explained in Nazirizadeh,

Y.: “Photonic crystal slabs for surface contrast enhancement in microscopy of transparent objects”, Optics Express, Vol. 20, Issue 13, pp. 14451-14459 (2012). Therefore, the content of this publication is incorporated by reference herein.

Further, the receptacle 4 of FIG. 4 comprises a calibration element 17 being arranged on the upper side of the bottom of the receptacle 4. The calibration element 17 allows a measurement of the transfer function of the data acquisition system 1 which in turn allows an improvement of the optical resolution.

FIG. 5 largely corresponds to FIG. 3 and additionally comprises the calibration element 17 as mentioned above.

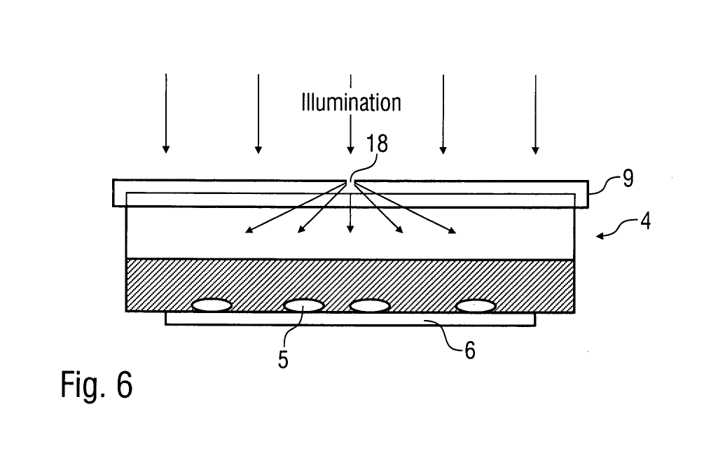

FIG. 6 shows a further modification of the receptacle 4 as shown in FIG. 2-5. Instead of the light source 8, the receptacle 4 comprises a hole 18 in the center of the cover 9, so that the sample 5 in the receptacle 4 is illuminated by ambient light through the hole 18.

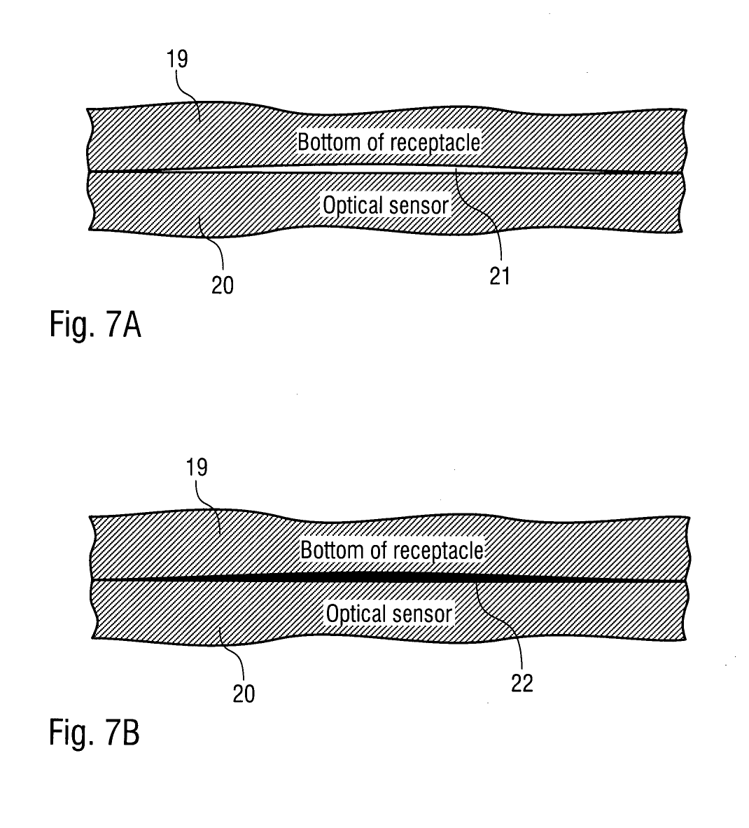

FIG. 7A shows a cross section through a bottom 19 of the receptacle 4 being arranged on a photosensitive surface 20 of the image sensor 6. The cross section shows that there is an air gap 21 between the bottom 19 of the receptacle 4 and the photosensitive surface 20 of the image sensor 6. However, the air gap 21 causes multiple reflections thereby impairing the quality of the images 2.

FIG. 7B shows an improvement of FIG. 7A, wherein the air gap 21 is filled with an immersion oil 22, so that multiple reflections are avoided thereby improving the quality of the images.

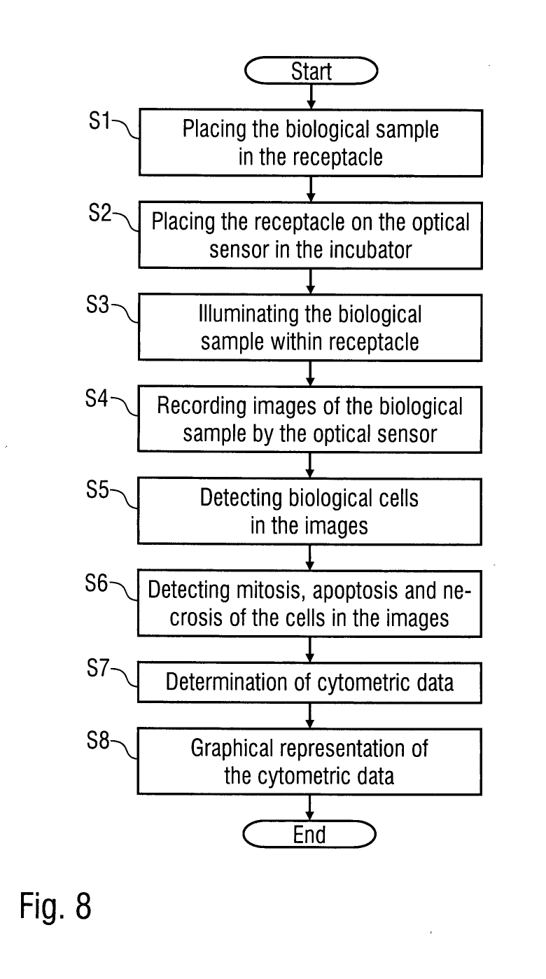

FIG. 8 shows a flow chart illustrating the operating method of the system as shown in FIG. 1.

In a first step S1, the biological sample 5 is placed in the receptacle 4 on the bottom of the receptacle 4.

In a next step S2, the receptacle 4 is placed on the photosensitive surface 20 of the image sensor 6 in the incubator, wherein the incubator is not shown in the drawings.

Then, the biological sample 5 in the receptacle 4 is illuminated in step S3 and the images 2 of the biological sample 5 are recorded by the image sensor 6 in step S4.

The evaluation unit 3 then detects biological cells in the images 2 in step S5.

In a following step S6 the evaluation unit 3 detects mitosis, apoptosis and necrosis of the cells in the images 2.

In another step S7, the evaluation unit 3 determines cytometric data relating to the cells shown in the images 2.

Finally, the cytometric data are graphically represented in step S8.

Although the invention has been described with reference to the particular arrangement of parts, features and the like, these are not intended to exhaust all possible arrangements of features and indeed many other modifications and variations will be ascertainable to those of skill in the art.

### LIST OF REFERENCE SIGNS

1 Image acquisition system

2 Images

3 Evaluation unit

4 Receptacle

5 Sample

6 Image sensor

7 Pressing mechanism

8 Light source

9 Cover

10 Actuator

11 Reflecting element

12 Light source

13 Light source

14 Upper polarization

15 Lower polarization filter

16 Optical waveguide structure

17 Calibration element

18 Hole in the cover

19 Bottom of the receptacle

20 Photosensitive surface of the optical sensor

21 Air gap

22 Immersion oil

1-19. (canceled)

20. A receptacle for receiving a sample during an optical analysis of the sample, wherein the receptacle comprises a bottom which is at least partially transparent so that the sample within the receptacle can be optically analyzed by an image sensor from below the bottom, wherein the bottom of the receptacle comprises a thickness of less than 500 μm.

21. The receptacle according to claim 20, wherein the thickness of the bottom of the receptacle is sufficiently small to allow gas diffusion through the bottom.

22. The receptacle according to claim 20, wherein
a) an upper polarization filter is arranged above the sample between the sample and a light source illuminating the sample from above,
b) a lower polarization filter is arranged below the sample between the sample and the image sensor viewing the sample from below,
c) an optical wave guide structure is arranged between the upper polarization filter and the lower polarization filter, and
d) the upper polarization filter and the lower polarization filter are aligned perpendicular to each other thereby restricting the light received by the image sensor from the light source to specific optical modes.

23. The receptacle according to claim 20, further comprising:
a) an upper color filter being arranged above the sample between the sample and a light source illuminating the sample from above, and
b) a lower color filter being arranged below the sample between the sample and the image sensor viewing the sample from below.

24. The receptacle according to claim 23, wherein
a) a wavelength of illumination from the light source is within a passband of the upper color filter, so that the illumination from the light source passes the upper color filter, and
b) a wavelength of light emitted by the sample in response to the illumination by the light source is within a passband of the lower color filter, so that the light emitted by the sample passes the lower color filter.

25. The receptacle according to claim 23, wherein
a) the upper side of the bottom of the receptacle is coated with a pH-sensitive fluorescent dye emitting light in response to the illumination by the light source, and
b) those parts of the pH-sensitive fluorescent dye not in contact with the sample emit light at an emission wavelength outside the passband of the lower color filter,
c) those parts of the pH-sensitive fluorescent dye in contact with the sample are pH-shifted by the sample thereby shifting the emission wavelength of the pH-sensitive fluorescent dye, wherein the shifted emission wavelength of the pH-sensitive fluorescent dye is within the passband of the lower color filter.

26. The receptacle according to claim 22, wherein
a) the upper polarization filter is arranged in a cover of the receptacle, and
b) the lower polarization filter is arranged in the bottom of the receptacle.

27. The receptacle according to claim 22, wherein
a) the upper color filter is arranged in a cover of the receptacle, and
b) the lower color filter is arranged in the bottom of the receptacle.

28. The receptacle according to claim 20, further comprising a calibration element for optical calibration.

29. The receptacle according to claim 20, wherein a light source is integrated in the receptacle for illuminating the sample within the receptacle from above.

30. The receptacle according to claim 29, wherein the receptacle comprises a cover, wherein the light source is arranged at least partially in the cover.

31. The receptacle according to claim 29, wherein the light source is point-shaped.

32. The receptacle according to claim 30, wherein the light source comprises a lamp, arranged in the cover of the receptacle.

33. The receptacle according to claim 30, wherein the light source comprises a hole in the cover of the receptacle.

34. The receptacle according to claim 29, wherein the light source comprises a reflecting element above the sample, and a lamp for illuminating the reflecting element from below, wherein the reflecting element is preferably shaped as a circle or as a half-sphere.

35. A system for optically analyzing a sample, comprising:
a) an image sensor comprising a photosensitive area with a plurality of photosensitive pixels, and
b) a receptacle for receiving the sample during analysis of the sample,
c) wherein the receptacle is arranged directly on the photosensitive area of the image sensor without any optical lens between the receptacle and the image sensor.

36. The system according to claim 35, further comprising a pressing mechanism for pressing the receptacle onto the photosensitive area of the image sensor for avoiding an air gap between the photosensitive area of the image sensor on the one hand and the bottom of the receptacle on the other hand.

37. The system according to claim 35, wherein any gap between the bottom of the receptacle and the photosensitive area of the image sensor is at least partially filled with a liquid.

38. The system according to claim 35, further comprising an actuator for moving the image sensor relative to the receptacle in a plane of the photosensitive area of the image sensor in order to increase an optical resolution of the measurement, wherein an amplitude of a relative movement is smaller than a distance between adjacent pixels of the image sensor.

39. The system according to claim 35, further comprising means for varying a direction of illumination of the sample within the receptacle.

40. The system according to claim 39, wherein the means for varying the direction of illumination comprises an array of lamps and/or mirrors, wherein the lamps and/or mirrors are located at different places above the sample for successively illuminating the sample from different angles.

41. The system according to claim 35, wherein the photosensitive area of the image sensor is larger than 200 mm².

42. The system according to claim 35, wherein a light flux passing through the sample is much smaller than the light flux passing through the sample in a light microscope so that a light exposure of the sample is reduced.

43. The system according to claim 35, further comprising an incubator, wherein the receptacle is arranged within the incubator.

44. The system according to claim 35, further comprising an evaluation unit for an images-based evaluation of images recorded by the image sensor, wherein the evaluation unit performs at least one of the following steps:
a) detection of biological cells in the image recorded by the image sensor,
b) tracking of a position of the biological cells within the image,
c) detecting apoptosis of the biological cells,
d) detecting necrosis of the biological cells,
e) detecting mitosis of the biological cells,
f) determination of an image entropy of the image recorded by the image sensor,
g) determination of cytometric data selected from the group consisting of:
g1) total number of the biological cells within the image,
g2) cell proliferation of the cells within the image,
g3) frequency of mitosis among the biological cells within the image,
g4) morphological parameters of the biological cells, and
g5) duration of a cell cycle.

45. The system according to claim 35, wherein the receptacle comprises a bottom which is at least partially transparent so that the sample within the receptacle can be optically analyzed by an image sensor from below the bottom, wherein the bottom of the receptacle comprises a thickness of less than 500 μm.
